(19) United States
(12) Patent Application Publication
(10) Pub. No.: US 2025/0367271 A1
(43) Pub. Date: Dec. 4, 2025

(54) COMPOSITIONS FOR TREATING ADDICTION

(71) Applicant: Curemark, LLC, Rye Brook, NY (US)

(72) Inventors: Joan M. Fallon, White Plains, NY (US); Matthew F. Heil, Sherman, CT (US)

(21) Appl. No.: 19/021,940

(22) Filed: Jan. 15, 2025

Related U.S. Application Data
(63) Continuation of application No. 16/499,988, filed on Oct. 1, 2019, now Pat. No. 12,226,464.
(60) Provisional application No. 62/483,856, filed on Apr. 10, 2017.

(51) Int. Cl. A61K 38/54; A61K 9/48; A61K 9/50; A61K 45/06; A61P 25/36

(52) U.S. Cl. CPC A61K 38/54; A61K 9/4808; A61K 9/4825; A61K 9/4891; A61K 9/5015; A61P 25/36; A61K 45/06

(57) ABSTRACT

A pharmaceutical composition for use in the treatment of the symptoms of an addiction is disclosed. The pharmaceutical composition contains, but is not limited to, digestive enzymes. The pharmaceutical composition may further contain a coating that surrounds a core of digestive enzymes in the form of coated particles. The pharmaceutical composition may also be encapsulated. The therapeutic agent may be manufactured by a variety of technologies.

### CROSS-REFERENCE

This application is a continuation of patent application Ser. No. 16/499,988, filed Oct. 1, 2019, which is a national stage entry of International Application No. PCT/US18/26841, filed on Apr. 10, 2018, which claims the benefit of U.S. Provisional Patent Application No. 62/483,856, filed on Apr. 10, 2017, which is incorporated by reference herein in its entirety.

### BACKGROUND

Addiction is one of the most costly public health problems in the United States. It is a progressive syndrome, which means that it increases in severity over time unless it is treated. Substance abuse is characterized by frequent relapse or return to the abused substance. Substance abusers often make repeated attempts to quit before they are successful.

In 1995 the economic cost of substance abuse in the United States exceeded $414 billion, with health care costs attributed to substance abuse estimated at more than $114 billion.

By eighth grade, 52% of adolescents have consumed alcohol, 41% have smoked tobacco, and 20% have smoked marijuana. Compared to females, males are almost four times as likely to be heavy drinkers, nearly one and a half more likely to smoke a pack or more of cigarettes daily, and twice as likely to smoke marijuana weekly. However, among adolescents these gender differences are decreasing. Although frequent use of tobacco, cocaine and heavy drinking appears to have remained stable in the 1990s, marijuana use increased.

In 1999, an estimated four million Americans over the age of 12 used prescription pain relievers, sedatives, and stimulants for “nonmedical” reasons during one month.

In the United States, 25% of the population regularly uses tobacco. Tobacco use reportedly kills 2 5 times as many people each year as alcohol and drug abuse combined. According to 1998 data from the World Health Organization, there were 1.1 billion smokers worldwide and 10,000 tobacco-related deaths per day. Furthermore, in the United States, 43% of children aged 2 to 11 years are exposed to environmental tobacco smoke, which has been implicated in sudden infant death syndrome, low birth weight, asthma, middle ear disease, pneumonia, cough, and upper respiratory infection.

### SUMMARY OF THE INVENTION

Provided herein are methods of treating addiction by administering to the subject pharmaceutical compositions that comprise digestive enzymes. In some instances of such methods, treatment reduces an amount of ΔFosβ in the subject.

Provided herein is a method for treating a subject having an addiction, comprising administering to the subject a therapeutically effective amount of a pharmaceutical composition that comprises digestive enzymes to the subject, whereby the addiction is treated, and wherein the addiction is a drug addiction, an alcohol addiction, or a nicotine addiction. In some instances, administration of the pharmaceutical composition reduces an amount of ΔFosβ in the subject compared to prior to administration of the pharmaceutical composition. In certain instances, the digestive enzymes comprise an amylase, a lipase, and a protease.

Provided herein is a method of reducing an elevated amount of ΔFosβ in a subject having an addiction, comprising administering to the subject a therapeutically effective amount of a pharmaceutical composition that comprises digestive enzymes, wherein the transcription factor is elevated in the subject due to the addiction compared to a subject without the addiction. In certain instances, the digestive enzymes comprise an amylase, a lipase, and a protease.

Provided herein is a method of decreasing an amount or length of dendritic spines in nerve cells of a subject having an addiction, comprising administering to the subject a therapeutically effective amount of a pharmaceutical composition that comprises digestive enzymes, thereby decreasing an amount of dendritic spines in nerve cells of the subject to an amount that is lower than prior to administration of the pharmaceutical composition. In certain instances, the digestive enzymes comprise an amylase, a lipase, and a protease.

Provided herein is a method of treating an addiction in a subject in need thereof, comprising administering to the subject one or more doses of a pharmaceutical composition that comprises digestive enzymes; wherein the digestive enzymes that comprise a protease, an amylase and a lipase; wherein a dose of the pharmaceutical composition at one point of administration to the subject comprises from about 650,000 units of the protease to about 1.5×10⁶ units of the protease.

In such methods, in some instances, the digestive enzymes in the pharmaceutical composition further comprise an enteric coating or a lipid coating.

In some aspects, a pharmaceutical composition described herein is encapsulated.

The pharmaceutical composition is a dosage form can be, for example, a capsule, a tablet, or a sachet.

It will be understood that, when referencing a “dose” of the pharmaceutical composition, the dose refers to about 4, about 5, about 6, about 7, about 8, or about 9 capsules, tablets or sachets.

The pharmaceutical composition may be administered to the subject from about one to about three times per day. In some instances, the pharmaceutical is administered to the subject three times a day. In other instances, the pharmaceutical is administered to the subject two times a day. In other instances, the pharmaceutical is administered to the subject one time per day. It will be understood that if a subject demonstrates a reduction in the addiction, then the amount of the pharmaceutical composition to be administered to the subject at each dose and/or the number of administrations per day may be reduced.

The pharmaceutical composition may be administered to the subject with a meal.

Provided herein is a method of treating an addiction in a subject in need thereof, comprising administering to the subject one or more doses of a pharmaceutical composition that comprises encapsulated coated digestive enzyme particles; wherein the coated digestive enzyme particles comprise (i) a core that comprises digestive enzymes that comprise a protease, an amylase and a lipase, and (ii) a coating; wherein a dose of the pharmaceutical composition comprises from about 4 to about 9 capsules of the encapsulated coated digestive enzyme particles; wherein from about 4 to about 9 capsules are administered to the subject three times a day with meals for at least 12 weeks; and wherein from about 650,000 units of the protease to about 1.5×10⁶ units of the protease are administered to the subject with each meal.

The subject may be addicted to one or more drugs, one or more types of alcohol, one or more types of nicotine, or a combination thereof.

In one instance, the addiction comprises a drug addiction. A drug as disclosed herein refers to, for example, an opiate, cocaine, crack, ecstasy, PCT, LSD, or a combination thereof.

Opiates include, but are not limited to, a natural opiate, a synthetic opiate, or a combination thereof.

Natural opiates include, but are not limited to, opium, morphine, codeine, heroin, and a combination thereof.

Synthetic opiates include, but are not limited to, Dilaudid (hydromorphone hydrochloride), Demerol, Oxycodone, Vicodin, Fentanyl, Methadone, and a combination thereof.

The amylase, in some instances, is present in the pharmaceutical composition in an amount of from about 120,000 to about 370,000 U.S.P. units/unit dose.

The protease, in some instances, is present in the pharmaceutical composition in an amount of from about 130,000 to about 165,000 U.S.P. units/unit dose.

The lipase, in some instances, is present in the pharmaceutical composition in an amount of from about 17,000 to about 60,000 U.S.P. units/unit dose.

A total protease and a total lipase in the pharmaceutical composition in U.S.P. units are present in a ratio of protease to lipase of from about 1:1 to about 20:1.

A total protease and a total lipase in the pharmaceutical composition in U.S.P. units are present in a ratio of protease to lipase of from about 4:1 to about 10:1.

A total protease and a total lipase in the pharmaceutical composition in U.S.P. units are present in a ratio of protease to lipase of 4:1, 5:1, 6:1, 7:1, 8:1, 9:1, or 10:1.

A total protease and a total amylase in the pharmaceutical composition in U.S.P. units are present in a ratio of protease to amylase of 1:0.1 to 1:10.

The pharmaceutical composition may be manufactured using any suitable technology including those selected from the group consisting of direct compression, dry granulation, wet granulation, and a combination thereof.

The pharmaceutical composition, in some instances, comprises coated digestive enzyme particles.

The pharmaceutical composition, in some instances, comprises coated digestive enzyme particles that are encapsulated.

Coated digestive enzyme particles comprise (i) a core that comprises the digestive enzymes and (ii) a coating.

In certain instances, the coating comprises an enteric coating.

In other instances, the coating comprises a lipid.

A lipid can comprise a food grade lipid such as, for example, a sorbitan monostearate, a sorbitan tristearate, or a calcium stearoyl lactylate.

A lipid can comprise a pharmaceutical grade lipid such as, for example, a soybean oil that is fully hydrogenated (fully-hydrogenated soybean oil).

A lipid can comprise one or more monoglycerides, one or more diglycerides, one or more triglycerides, fatty acids, esters of fatty acids, phospholipids, or a combination thereof. In some instances, the lipid comprises one or more monoglycerides. In some instances, the lipid comprises one or more diglycerides. In some instances, the lipid comprises one or more triglycerides. In some instances, the lipid comprises monoglycerides and diglycerides. In some instances, the lipid comprises monoglycerides and triglycerides. In some instances, the lipid comprises diglycerides and triglycerides. In some instances, the lipid comprises monoglycerides, diglycerides and triglycerides.

A lipid can comprise a hydrogenated lipid, a saturated lipid, a partially saturated lipid, or a combination thereof.

In some instances, the lipid comprises a soy lipid. A soy lipid can comprise a hydrogenated soy lipid.

Where the lipid comprises the esters of fatty acids, the esters of fatty acids are selected from the group consisting of acetic acid esters of mono- and diglycerides, citric acid esters of mono- and diglycerides, lactic acid esters of mono- and diglycerides, polyglycerol esters of fatty acids, propylene glycol esters of fatty acids, and diacetyl tartaric acid esters of mono- and diglycerides.

The lipid can comprise comprises a hydrogenated castor wax or a hydrogenated carnauba wax.

The lipid can comprise an animal lipid or a vegetable lipid such as, for example, a palm kernel oil, a soybean oil, a cottonseed oil, a canola oil and a poultry fat.

The pharmaceutical composition can be administered to the subject orally.

The pharmaceutical composition can be a dosage formulation selected from the group consisting of a pill, a tablet, a capsule, a sprinkle, a sachet, and a combination thereof. In some instances, the capsule comprises a microcapsule, a mini-capsule, a time released capsule, or a combination thereof. In some instances, the tablet comprises a minitab.

The coated digestive enzyme particles can, in some instances, be encapsulated in a gelatin capsule or a hydroxypropyl methylcellulose (HPMC) capsule. An HPMC capsule can comprise a size 00 capsule or a size 0 capsule.

Where the pharmaceutical composition comprises a capsule, a single capsule comprises a protease activity of from about 135,000 United States Pharmacopeia (U.S.P.) units/mg to about 150,000 U.S.P. units/mg.

In some instances, the subject is administered one or more doses of the pharmaceutical composition.

A dose of the pharmaceutical composition can comprise from about 4,000 to about 9,000 mg of the digestive enzymes.

The digestive enzymes can be present in the coated particles in an amount of from about 70% to about 90% by weight, or from about 75% to about 85% by weight. In some of the disclosed methods, the digestive enzymes can be present in the coated particles in an amount of about 70% by weight. In some of the disclosed methods, the digestive enzymes can be present in the coated particles in an amount of about 72.5% by weight. In some of the disclosed methods, the digestive enzymes can be present in the coated particles in an amount of about 75% by weight. In some of the disclosed methods, the digestive enzymes can be present in the coated particles in an amount of about 77.5% by weight. In some of the disclosed methods, the digestive enzymes can be present in the coated particles in an amount of about 80% by weight. In some of the disclosed methods, the digestive enzymes can be present in the coated particles in an amount of about 82.5% by weight. In some of the disclosed methods, the digestive enzymes can be present in the coated particles in an amount of about 85% by weight. In some of the disclosed methods, the digestive enzymes can be present in the coated particles in an amount of about 875% by weight. In some of the disclosed methods, the digestive enzymes can be present in the coated particles in an amount of about 90% by weight.

In one instance, at least 90% of the coated particles are from about 105 μm to about 425 μm in size. In one instance, at least 75% of the coated particles are from about 180 μm to about 425 μm in size. In one instance, less than about 20% of the coated particles are capable of being sieved through about 150 μm mesh. In one instance, less than about 15% of the coated particles are capable of being sieved through about 150 μm mesh.

In such methods, the ΔFosβ can comprise ΔFosβ. An amount of the ΔFosβ after administration of the pharmaceutical composition to the subject can be at least about 2-fold, at least about 3-fold, at least about 4-fold, at least about 5-fold, at least about 10-fold, at least about 50-fold, or at least about 100-fold lower than the amount of the ΔFosβ prior to administration of the pharmaceutical composition.

An amount of the ΔFosβ after administration of the pharmaceutical composition to the subject can be at least about 2%, about 5%, about 10%, about 15%, about 20%, about 25%, about 30%, about 35%, about 40%, about 45%, about 50%, about 55%, about 60%, about 65%, about 70%, about 75%, about 80%, less than the amount of the ΔFosβ prior to administration of the pharmaceutical composition.

The pharmaceutical composition can be packaged in a blister pack, a trilaminar foil pouch, or a bottle. A bottle is, for example, a glass bottle or a plastic bottle sealed with foil. The pharmaceutical composition can packaged in a blister pack that comprises a foil backing.

Administration of the pharmaceutical composition to the subject can reduce a craving associated with the addiction. The method can reduce the length of the craving, the severity of the craving, or the length of the craving and the severity of the craving.

Administration of the pharmaceutical composition to the subject can reduce impulsivity associated with the addiction. The method can reduce the length of the impulsivity, the severity of the impulsivity, or the length of the impulsivity and the severity of the impulsivity.

Administration of the pharmaceutical composition to the subject can reduce a lack of impulse control associated with the addiction.

Administration of the pharmaceutical composition to the subject can reduce a craving associated with the addiction and can reduce a lack of impulse control associated with the addiction.

Administration of the pharmaceutical composition to the subject can reduce a craving associated with the addiction and reduces impulsivity associated with the addiction.

Administration of the pharmaceutical composition to the subject impulsivity associated with the addiction and reduces a lack of impulse control associated with the addiction.

In some aspects, a blood sample can be obtained from the subject and tested for a level of phenylalanine prior to commencement of treatment, and at one or more time points during and/or after treatment. The blood level of phenylalanine in the subject can be compared to a normal blood level of phenylalanine from a healthy, non-addicted subject. In some instances, the normal blood level of phenylalanine from a healthy, non-addicted subject comprises from about 2 mg/dL to about 6 mg/dL of phenylalanine.

In some instances, the normal blood level of phenylalanine from a healthy, non-addicted subject comprises from about 120 micromolar to about 360 micromolar of phenylalanine.

Provided herein is a pharmaceutical composition for treatment of an addiction in a subject in need thereof, wherein the pharmaceutical composition comprises encapsulated, coated digestive enzyme particles, wherein the coated digestive enzyme particles comprise (i) a core that comprises digestive enzymes that comprise a protease, an amylase and a lipase, and (ii) a coating. In some instances, the coating comprises a lipid coating. In other instances, the coating comprises an enteric coating.

A dose of the pharmaceutical composition can comprise from about 4 to about 8 capsules and each capsule can comprise from about 135,000 United States Pharmacopeia (U.S.P.) units/mg to about 150,000 U.S.P. units/mg. of protease activity. A single administration time point of the pharmaceutical composition can, therefore, comprise from about 650,000 U.S.P. units to about 1.5×10⁶ U.S.P. units. of protease activity.

The pharmaceutical composition may be formulated to be administered to the subject three times a day, and the pharmaceutical composition to be administered at each dosage event comprises from about 4,000 mg to about 9,000 mg of the digestive enzymes.

In one aspect, provided herein is the use of any of the pharmaceutical compositions described herein, in the formulation of a medicament for the treatment of an addiction in a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates essential and non-essential amino acids.

FIG. 2 depicts proliferation of dendritic splines as a result of repeated drug use.

FIG. 3 illustrates a non-limiting example, of a mechanism of action of treatment of an addiction following administration of a pharmaceutical composition to a subject.

### DETAILED DESCRIPTION OF THE INVENTION

It is to be understood that this application is not limited to particular formulations or process parameters, as these may, of course, vary. It is also to be understood that the terminology used herein is for the purpose of describing particular embodiments only and is not intended to be limiting. Further, it is understood that a number of methods and materials similar or equivalent to those described herein can be used in the practice of the present inventions.

The terms “about” and “approximately” includes equal to, and a range that takes into account experimental error in a given measurement. As used herein, the terms “about” and “approximately,” when used to modify a numeric value or numeric range, indicate that deviations of plus or minus (±) 10, 9, 8, 7, 6, 5, 4, 3, 2 or 1% or anywhere in-between. For example, a value may be 0.2%, 0.5%, 1%, 2.5%, 5%, 7.5%, or 10% (or any integer between about 0.2% and 10%) above or below the value or range remain within the intended meaning of the recited value or range. In some instances, the term “about” refers to plus or minus (±) 2%, 1.5%, 1%, 0.5% or 0.2% of the indicated value.

As used in this specification and the appended claims, the singular forms “a”, “an”, and “the” include plural references unless the context clearly dictates otherwise. Thus, for example purposes only, references to “a method” include one or more methods, and/or steps of the type described herein and/or which will become apparent to those persons skilled in the art upon reading this disclosure.

As used herein, “addiction” refers to a dependence on a behavior or substance that a subject is powerless to stop or is difficult to stop. The term has been partially replaced by the word “dependence” for substance abuse. Addiction includes mood-altering behaviors or activities. There is a growing recognition that many addicts can be addicted to more than one substance.

The invention relates to the use of digestive enzymes in the treatment of a drug addiction, an alcohol addiction, or a combination thereof.

According to the Office of National Drug Control Policy, there are over 3 million long-term cocaine users in the United States. There are currently no medications approved to treat cocaine addiction. Cocaine addiction can produce significant morbidity and mortality. There is a large need for effective treatment that can produce changes in overall craving and subsequently reduce cocaine use.

According to the National Survey on Drug Use and Health (NSDUH) adults aged 18 to 25 years have a higher rate of current cocaine use than any other age group, with 1.5 percent of young adults reporting cocaine use in the past month. Overall, men report higher rates of current cocaine use than women. Further, data from the 2008 Drug Abuse Warning Network (DAWN) report showed that cocaine was involved in 482,188 of the nearly 2 million visits to emergency departments for drug misuse or abuse. This translates to almost one in four drug misuse or abuse emergency department visits (24 percent) that involved cocaine.

The liver is one of the most injured organs in cases of alcoholism and drug abuse. The reason is because the liver is part of the digestive system and is used by the body to filter out and store toxins. When it is overtaxed, the liver can become fatty or damaged or both. It is critical that those going through drug or alcohol rehabilitation emphasize liver healing.

Alcohol blocks the absorption and breakdown of nutrients by damaging the cells lining the stomach and intestines, and by decreasing the amount of digestive enzymes secreted by the pancreas. For reasons that aren't yet known, the pancreas can become inflamed and leak digestive enzymes, which then attack the pancreas itself.

Amino acids have long been described as the building blocks of proteins. Amino acids are classified as essential, non-essential and semi-essential. Essential amino acids are those that the body cannot synthesize or otherwise obtain endogenously in both children and adults. These essential amino acids must be obtained exogenously by eating a protein and breaking the protein down into its component amino acids through enzymatic hydrolysis. Semi-essential amino acids are those that young children must obtain exogenously since they cannot be synthesized or procured endogenously in the pediatric population. Semi-essential amino acids can be procured or synthesized endogenously in adults. Non-essential amino acids are those that the body can procure or synthesize endogenously in both adults and children.

Amino acids are cleaved from the ingested protein as mono-, di-, tri-tetra- and higher peptide fragments. Amino acid pool depletion and the lack of the building blocks for new proteins represent one way in which diminished numbers of amino acids, especially essential amino acids

Phenylalanine has been found to play in the pathophysiology of addiction especially cocaine addiction quite possibly by the delta Fosβ (ΔFosβ) theory of brain change resulting in addiction.

Emerging evidence demonstrates that amino acids are not only involved in protein synthesis but also play a significant role in other important neurological functions. For example those essential amino acids cleaved by the digestive enzyme chymotrypsin, phenylalanine and tryptophan, are necessary components of the body's synthesis of dopamine and serotonin respectively.

### Physiology

Classical thinking with respect to amino acids identifies them as the building blocks of new proteins synthesized by the body. Essential amino acids need to be ingested in the form of protein and are cleaved from that protein. They cannot be obtained any other way or synthesized by the body. Further the administration of these essential amino acids directly into the GI tract results in poor uptake and utilization. The semi-essential act similarly as essential amino acids in small children and the non-essential amino acids can be synthesized by the body without any additional input from ingested exogenous proteins.

While this classical view has been held for decades, we now know the demarcation between essential and non-essential is not as well defined as previously believed. For example, the endogenous synthesis of many non-essential amino acids is under the direct control of essential amino acids. So, while in theory a non-essential can be synthesized, an essential amino acid may be necessary for that synthesis to occur.

Further, for example, the essential amino acid methionine serves as the initiation codon at the beginning of all protein synthesis in humans. This is also a very important amino acid with respect to RNA replication. Leucine is the only amino acid required to optimally stimulate muscle protein synthesis. Both methionine and leucine and the processes they enable are necessary in childhood for growth and repair.

In addition to their role in neurotransmitters, regulators of protein degradation, nitrogen physiology and catabolism, amino acids are emerging as important regulators of gene expression. This role was only first described in the literature over the last 15 years.

Starvation of certain amino acids can actually determine the up-regulation or down-regulation of specific genes. For example, gene expression is strongly induced in response to methionine starvation. The CHOP gene is of particular interest because of its role in controlling neural pruning in the developing brain through apoptosis.

There are a wide variety of mechanisms by which amino acids can affect regulation of gene expression. Experiments conducted in yeast have shown that regulation can proceed through very specific forms of control such as those reliant upon end products of enzymes associated with a particular biosynthetic pathway. For example, the synthesis of leucine is controlled by the transcriptional activator Leu3p in response to the availability of leucine.

An additional example of regulation is found in the deprivation of certain amino acids or stress such as that from heat shock. In this instance, a lack of specific amino acids causes the corresponding tRNAs to accumulate, which in turn leads to an increased production of a transcriptional regulator.

There have been well over 500 genes identified that can be affected by the presence of absence of specific amino acids. Through the outside availability of food sources or the ability of the body to utilize those sources; this relatively new finding in gene regulation represents an important source of epigenetic influence affecting the course of disease pathology.

The pathophysiology section below describes new theories of cocaine addiction and the role of the brain transcription factor ΔFosβ being interdependent with the essential amino acid phenylalanine. The changes in ΔFosβ after cocaine use result in elongation of the dendritic spines in the presence of a depleted source of phenylalanine.

The role of amino acid and especially amino acid pool depletion especially of essential amino acids, is no longer relegated to simply “the building blocks of protein”.

### Pathophysiology

Deciphering the genetic code over the last 3 decades has revealed the presence of three separate stop codons and a single start codon in DNA. The start codon is so designated because this is the point where the information for creating any particular protein begins in the genome. This information is initially stored in DNA and consists of a sequence of purine and pyrimidine bases. The code sequence is always the same and it specifically codes for the amino acid methionine. Thus, methionine is the first amino acid contained in all proteins created by the body. By virtue of its status as the initial amino acid for all protein synthesis, methionine is absolutely essential for life.

The lack of amino acids can affect multiple processes in the human body, including but not limited to the formation of neurotransmitters, epigenetic influences, protein catabolism and anabolism, multiple levels of transcription and persistence of addictive behaviors. The fact that the lack or presence of amino acids can have an epigenetic effect and potentially turn on and off gene expression adds to the importance of the availability of a complete amino acid pool. While the complex balance of amino acids is necessary for life, there are basic tenets of that balance that hold true for all human proteomics:

An Amino Acid Response system (AAR) has been identified; this system signals and identifies deficiencies and imbalances in amino acids in the human body. Dietary intake of amino acids is typically not balanced to exactly match the body's demands for various amino acids. Amino acids taken via the diet must be chemically modified and rearranged to provide adequate levels of all the amino acids needed. Essential amino acids can only be procured from ingested protein; they cannot be manufactured in the body. Non-essential amino acids can be manufactured in the body, however, in some cases they are under the direct control of essential amino acids.

Amino acid absorption pathways in the gastrointestinal mucosa are configured to transport di and tri-peptides, not single amino acids. Supplementation with single amino acids will not be sufficient for the replacement of amino acids due to a lack of protein ingestion. In humans, unlike other mammals, levels of protein in the diet cannot induce digestive proteases to pathologic levels. Amylases and to some degree lipases can be induced by the level of carbohydrate and fat in the diet. There are a significant number of pathways in the body for balancing the pool of amino acids, both for synthesis and for degradation. The number of enzymes and co-enzymes necessary for each creates a great potential for disease when there is a deficiency in the pathway.

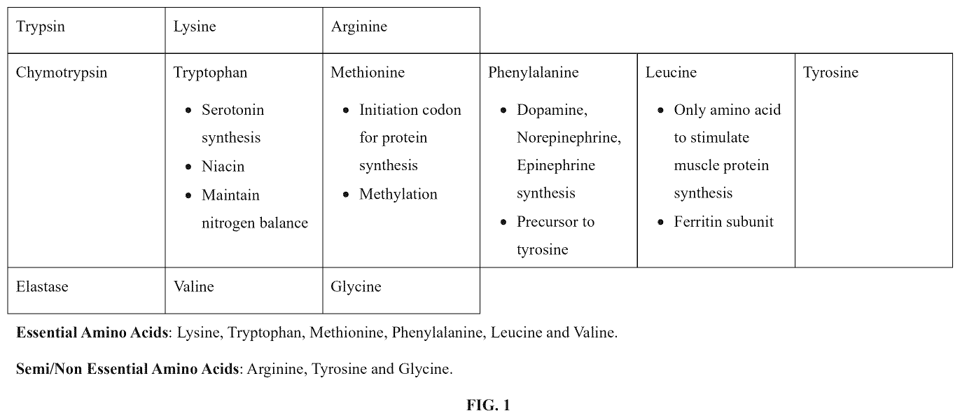

The genetic influences of the amino acids may account for certain disease states. Disruption of the amino acid sequence, even by just one amino acid, can have profound consequences for growth and development. The consequences may be physiological, cognitive, psychological and behavioral. Some of these are inheritable and many are fatal. The essential and non-essential amino acids have the ability to control protein synthesis, gene regulation, nitrogen balance and neurotransmission, among other things. The importance of the amino acid supply and the ability to synthesize non-essential and to obtain essential amino acids from the diet is paramount. The inability of the body to directly synthesize essential amino acids and the fact that the essential amino acids play a role in the synthesis of non-essential amino acids, as in the case of asparagine which is under the control of methionine (an essential amino acid, see FIG. 1), underscores the importance of the enzyme's activity and its ability to aid in the breakdown of protein and supply the body's amino acid pool.

The utilization and absorption of amino acids and the preferential uptake in the gastrointestinal system (specifically in the duodenum), is accomplished by the absorption of di- and tripeptides through the mucosa. There appears to be a preferential absorption (in volume and rate) of di and tri-peptides over both single amino acids and tetra and higher peptides. This is an important aspect of enzyme replacement utilization for those who lack chymotrypsin, or have an abnormally low level of fecal chymotrypsin (FCT). For children presenting with low/pathological levels of chymotrypsin, the need to deliver di- and tri peptides to the small intestinal mucosa for absorption is paramount.

Amino acid channels can be established in the small intestines where the specific breakdown of protein into di and tri-peptides occurs, but also, the amino acids are preferentially and more expeditiously absorbed in those forms, as evidenced by the 2-3 times faster absorption in the form of di and tri-peptides.

The absorption channels that allow for the maximized absorption of di and tri-peptides in the mucosal wall of the small intestine do not support the administration of single amino acids, which are in many dietary supplements. These single amino acids are mainly destroyed by the gastric acid of the stomach. Further, these absorption channels are the preferential way in which these di and tri-peptides are absorbed.

A large subgroup of children have been found to exhibit low levels of fecal chymotrypsin, thus, signaling the potential for the lack of protein digestion as chymotrypsin is one of the main protease enzyme in the human body. The inability to induce pathological levels pointed to the fact that this sub group may actually have a deficit in protein digestion. As chymotrypsin cleaves phenylalanine, tryptophan methionine and leucine, the indications for neurological implications of the potential lack of amino acids in the generalized amino acid pools was profound.

The lack of enzyme and the connection to neurological function cannot be ignored. It has been reported in the literature that children with low levels of tryptophan may also have low amounts of serotonin. Serotonin is a neurotransmitter that is found in the GI tract (80%), brain and platelets in humans and thought to be involved in the brain's control over behavior. Behaviors such as appetite, mood, aggression, sleep, memory, cognition, motor and sexual function have been cited as potential targets for serotonin's influence on behavior. Further, it may play a role in neuroendocrine signaling.

Deficiencies in serotonin have been linked to depression, increased appetite and other disorders. Since it has been linked to depression, there are a large number of antidepressants that target serotonin metabolism. One of these classes of drugs, the serotonin reuptake inhibitors (SSRI's), prevents the body from reabsorbing serotonin so that it can remain in the brain and remain available for the synapses.

The emergence of evidence with respect to cocaine addiction and the role of ΔFosβ that builds up in the brains of cocaine addicts is implicated in the persistence of cocaine addiction. In some instances, a subject to be treated with a method described herein comprises a subject addicted to a natural opiate, a synthetic opiate, or a combination thereof. Non-limiting examples of natural opiates include, but are not limited to, opium, morphine, codeine, heroin, etc., or a combination thereof. Synthetic opiates, on the other hand, are man-made in a laboratory and are most often used to treat chronic or severe pain. Non-limiting examples of synthetic opiates include, but are not limited to, Dilaudid, Demerol, Oxycodone, Vicodin, Fentanyl, Methadone, etc., or a combination thereof. In other instances, a subject to be treated with a method described herein comprises a subject addicted to crack, ecstasy, PCT, LSD, etc., or a combination thereof.

The presence of cocaine in the body of a subject can produce increased utilization of dopamine at the sites of dopamine transporters. The pleasure reward mechanism, loss of control, and compulsive behaviors are controlled by the limbic system which contains cells that are highly responsive to dopamine. These responsive cells are designed to trigger pleasurable responses that both make us feel good and want to repeat the experience. In some instances the drive for sexual pleasure and gratification can promote a desire to mate and result in species survival. On the other hand, the same mechanism can help to keep us repeating pleasurable yet destructive behaviors such as those seen in addiction.

ΔFosβ is a unique transcription factor that is derived from the fosB gene via alternative splicing and is known, therefore, as a genetic transcription factor.

One potentially key type of cocaine-related change that appears to last for many months after the last cocaine exposure, and perhaps longer is an alteration in the physical structure of nerve cells in the NAc. Chronic cocaine exposure causes these cells to extend and sprout new offshoots on their dendrites. Dendrites are the branch-like fibers that grow out from nerve cell bodies and collect incoming signals from other nerve cells. Just as a bigger antenna picks up more radio waves, more dendrite branches in the NAc theoretically will collect a greater volume of nerve signals coming from other regions—for example, the hippocampus, amygdala, and frontal cortex.

Because cocaine addiction decreases stores of phenylalanine, elongation of the dendritic spines occurs in the presence of a depleted source of phenylalanine. The converse is true in the case of PKU (phenyl ketone urea) where there is a buildup of phenylalanine, causing the dendrites to become stubs. Normal health subjects have baseline levels of phenylalanine of from about 2 mg/dL to about 6 mg/dL, or from about 120 micromolar to about 360 micromolar. It would be understood that a decreased store of phenylalanine can be 2-fold or more compared to the normal baseline.

The formation of the ΔFosβ from the Fosβ in the nucleus accumbens may occur through a “Leucine Zipper” mechanism where Leucine is found at every 7^{th} amino acid position. The elongation of the dendritic spines found in the presence of the ΔFosβ is regulated by the presence or absence of phenylalanine. The addition thereby results in an altered ratio of leucine/phenylalanine.

ΔFosβ is naturally present in small quantities in the cells of the Nucleus Accumbens in the brain, and chronic cocaine exposure causes it to accumulate to high levels. It has been postulated by some that ΔFosβ may play a role as a molecular “switch” in the transition from drug abuse to addiction, mainly for the following reasons.

Once created, a molecule of ΔFosβ lasts for 6 to 8 weeks before breaking apart chemically. Therefore, each new episode of cocaine abuse exacerbates the buildup of ΔFosβ that has accumulated from all previous episodes during roughly 2 months. If someone is abusing cocaine daily, the levels of ΔFosβ will be extremely elevated all the time.

ΔFosβ causes the elongation of and formation of dendritic spines, which may induce and maintain cocaine cravings; in the presence of phenylalanine, the elongation recedes to a normal state.

Mice with elevated ΔFosβ exhibit a set of behaviors that correspond to human addictive behaviors, while mice with normal levels of ΔFosβ do not. Conversely, blocking the buildup of ΔFosβ in mice during a regimen of cocaine exposure reduces these behaviors.

Chronic administration of cocaine increase ΔFosβ in several brain regions, most specifically the nucleus accumbens (NAc). Additionally, ΔFosβ also builds up in such areas as the frontal cortex and amygdala. The accumulations of ΔFosβ in the frontal cortex and the amygdala are much smaller than those that cocaine causes in the NAc.

ΔFosβ may cause more than 25 percent of chronic cocaine-induced changes in gene expression in the NAc—a finding that highlights the dominant role of this transcription factor in mediating cocaine's genetic effects in the brain. One of the genes stimulated by ΔFosβ is an enzyme, cyclin-dependent kinase-5 (CDK5), which promotes nerve cell growth. This finding sheds light on mechanisms underlying cocaine's very long-lasting effects on the brain.

Because ΔFosβ is induced in brain specifically by and remains in these brain regions for long periods of time, ΔFosβ may act as a sustained “molecular switch” that first initiates and then maintains some of the long-term adaptations of the brain in response to chronic perturbations.

Essential Amino Acids as well as non-essential amino acids play important roles in neurological function by directly affecting gene expression. The role of amino acids in gene expression opens up new doors to the role of epigenetics and disease such as addiction.

One key type of cocaine-related change that appears to last for many months after the last cocaine exposure, and perhaps longer is an alteration in the physical structure of nerve cells in the NAc. Chronic cocaine exposure causes these cells to extend and sprout new offshoots on their dendrites. Dendrites are the branch-like fibers that grow out from nerve cell bodies and collect incoming signals from other nerve cells. Just as a bigger antenna picks up more radio waves, more dendrite branches in the NAc theoretically will collect a greater volume of nerve signals coming from other regions—for example, the hippocampus, amygdala, and frontal cortex.

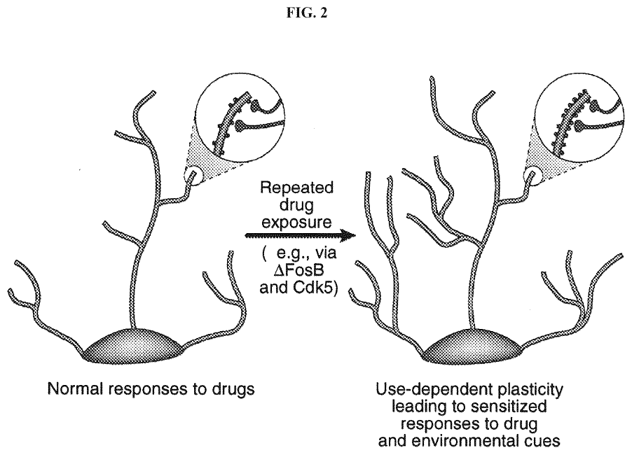

This elongation of the dendritic spines occurs in the presence of a depleted source of phenylalanine. The converse is true in the case of PKU (phenylketonuria) where there is a buildup of phenylalanine, causing the dendrites to become stubs. FIG. 2 illustrates proliferation of dendritic spines as a result of repeated drug use.

Emerging evidence demonstrates that amino acids are not only involved in protein synthesis but also play a significant role in other important neurological functions. Of the four essential amino acids cleaved by chymotrypsin, phenylalanine and tryptophan are necessary components of the body's synthesis of dopamine and serotonin, respectively.

The hypodopamanergic state, which has been hypothesized in cocaine addiction, may be amenable to treatment with a pharmaceutical composition described herein. The administration of the pharmaceutical composition can, in some instances, enhance the cleavage of phenylalanine and thereby increase the pool of this essential amino acid, the building block of dopamine. See, e.g., FIG. 1.

### Coated Digestive Enzyme Particles and Formulations

Provided herein are pharmaceutical compositions for use in an addiction treatment program for an active alcoholic and/or a drug addict. The pharmaceutical compositions may also be utilized to keep someone at risk for becoming an addict or it may be used to prevent someone from relapsing into addiction.

The pharmaceutical compositions may also be utilized for those who are deemed at risk for addiction due to family history or other historical events, such as severe stress or other factors placing the subject at risk.

As used herein, a “drug” refers to an addictive drug including, but not limited to, nicotine, cocaine, crack, heroin, PCP, LCD, ecstasy, or any other addictive drug.

As used herein, an “alcohol” refers to any type of alcohol including, but not limited to wine, beer, or a hard alcohol such as, for example, whisky, gin, moonshine, vodka, rum, etc.

As used herein, a “polyaddict” refers to a subject that is addicted to one or more drugs, one or more types of alcohol, or a combination thereof.

In one embodiment, a pharmaceutical composition that comprises digestive enzymes is formed into a dosage formulation containing a therapeutically effective amount of one or more proteases, one or more amylases, one or more lipases, or a combination thereof. In one non-limiting instance, the one or more proteases comprise chymotrypsin, trypsin, or a combination of chymotrypsin and trypsin.

In another embodiment, a pharmaceutical composition to be administered to a subject comprises a protease, an amylase and a lipase. In one non-limiting instance, the protease comprises chymotrypsin, trypsin, or a combination of chymotrypsin and trypsin.

The nature of the human digestive tract creates challenges for the delivery of digestive enzymes to patients with addictions to treatment with digestive enzymes. Multiple temperature and pH changes over the course of the digestive tract make specific delivery a necessity and a challenge. For instance, pH as low as 1 is encountered in the stomach, but rapidly increases to a more basic pH of 5-6 in the proximal small intestine. For example, generally the pH in the stomach is approximately 1.2, the pH in the duodenum is about 5.0 to 6.0; the pH in the jejunum is about 6.8, and the pH is about 7.2 in the proximal ileum and about 7.5 in the distal ileum. The low pH in the stomach which changes rapidly to a more basic pH of 5-6 in the proximal small intestines, call for a specific delivery method depending upon where the enzyme is to be delivered.

Delivery of digestive enzymes can also be challenging due to the rapid degradation and denaturing of enzymes at ambient room temperature, as well as the enhanced degradation and denaturing that can occur with high temperature, pressure, humidity and/or exposure to light. Moisture and heat together can quickly destabilize enzymes, reducing their effectiveness, and shortening shelf life, leading to inaccurate dosing. Denaturation or destabilization of the enzymes can reduce their effectiveness by reducing the dose of active enzymes to less than the amount needed for effective treatment. Alternatively, attempting to compensate for the denaturation or destabilization by increasing the dose to ensure an effective level of active enzyme, could risk an overdose or overfilling a capsule or other dosage form.

In one embodiment, the present invention includes digestive enzymes. In one instance, the digestive enzymes comprise a protease, an amylase, a lipase, or a combination thereof. In one non-limiting example, the digestive enzymes comprise one or more proteases, one or more amylases, and one or more lipases. In another non-limiting example, the digestive enzymes comprise a protease, an amylase, and a lipase. In certain instances, a pharmaceutical composition that comprises the digestive enzymes further comprises one or more enzymes selected from the group consisting of a sucrase, a cellulase, a maltase, papain, papaya, bromelain, or a combination thereof. In other instances, where a pharmaceutical composition comprises one or more proteases, the one or more proteases can be, for example, chymotrypsin, trypsin, bromelain, or a combination thereof. In one non-limiting embodiment, a pharmaceutical composition that comprises the digestive enzymes comprises a mixture of proteases such as, for example, a mixture of chymotrypsin and trypsin. In one non-limiting instance, digestive enzymes in a pharmaceutical composition can be provided as, for example, pancreatin. Such digestive enzymes can be provided as a crystalline form of pancreatin or as a solid form of pancreatin.

The digestive enzymes to be used in pharmaceutical compositions described herein can be animal enzymes, plant enzymes, microbial enzymes, synthetic enzymes, or a combination thereof. When the digestive enzymes are animal enzymes, they can be, in some instances, obtained from a mammal such as, for example, a pig pancreas. When the digestive enzymes are microbial enzymes, they can be, for example, fungal enzymes or enzymes from yeast. The digestive enzyme used in the present invention can be any combination of digestive enzymes of a type produced by the pancreas, including, but not limited to digestive enzymes from a pancreatic source or other sources. The scope of the invention is not limited to pancreatic enzymes of porcine origin. The enzyme may include one or more enzymes, and can also be plant derived, synthetically derived, recombinantly produced in microbial, yeast, or mammalian cells, and can include a mixture of enzymes from one or more sources. Digestive enzymes, can include, for example, enzymes from more or more sources mixed together. This includes, for example, the addition of single digestive enzymes to digestive enzymes derived from pancreatic sources in order to provide appropriate levels of specific digestive enzymes that provide more effective treatment for an addiction.

In one aspect, digestive enzymes described herein can be coated with an enteric coating. Enteric coatings include, for example, hypromellose phthalate, dimethicone 1000, dibutyl phthalate, or a combination thereof. A non-limiting example of an enteric coating for use herein includes, for example, ULTRASE®.

In other aspects, pharmaceutical compositions comprising coated digestive enzyme particles. These coated digestive enzyme particles contain a core that comprise digestive enzymes, and a coating that comprises a lipid. To protect and stabilize the digestive enzyme from unfavorable conditions such as penetration and decomposition, the digestive enzymes can be coated with an appropriate lipid or an appropriate blend of lipids.

In some aspects, a lipid coating comprises primarily one lipid. On other aspects, a lipid coating comprises a blend of lipids. Coated digestive enzyme compositions can, in some instances, exhibit improved (i.e., lengthened) shelf life in storage. The coatings in the digestive enzyme preparations create a barrier to degradation and denaturation, and allow more accurate levels of active enzymes to reach the treated individuals. The lipid coating of this invention provides a significant barrier to moisture, heat, humidity and exposure to light by allowing for a physical barrier as well as one that prevents and or reduces hydrolysis. The coated enzyme preparations undergo less hydrolysis as a result of protection from moisture in the environment by the lipid coating. As a result of the present invention, digestive enzymes are provided which can tolerate storage conditions (e.g., moisture, heat, oxygen, etc.) for long periods of time thus enabling extended shelf life. The coating of the digestive enzymes protects the enzyme from the environment.

It is a challenge to make digestive enzyme compositions that avoid the use of allergens, and other carriers, excipients, extenders, colorants, etc. that could potentially cause adverse symptoms or morbidity of subjects being treated. Furthermore, in very young children, administration of a composition with ease and tolerability is paramount.

It is another aspect of the present invention to prepare coated digestive enzyme particles without the use of extenders colorants, dyes, flow enhancers and other additives to reduce the potential for allergens and other sensitivity reactions. Surprisingly digestive enzyme particles containing lipases can be successfully with coating consisting essentially of only hydrogenated soy oil.

In addition, porcine digestive enzymes possess a significant odor and taste, similar to cured/smoked pork. This taste can be strong and offensive to some individuals taking enzyme replacement, and especially to children. The addition of a lipid coating provides significant taste masking to the enzyme preparation, which allows for the tolerance of taste, as the lipid coating is odorless and tasteless. The use of this method of taste masking which does not involve the use of color, dyes, perfumes, recipients, or other substances is preferable for the administration of medications, which have an unpleasant or undesirable taste and odor. In other embodiments, coated digestive enzyme preparations with improved taste and smell are prepared.

In some non-limiting instances, the coatings on the digestive enzyme particle cores can be continuous. By “continuous”, it is meant that the digestive enzymes are, generally, uniformly protected by the coating. The coating provides protection of the digestive enzymes from conditions such as moisture, temperature, and conditions encountered during storage.

In addition, the coating also provides controlled release of the digestive enzymes. The emulsification properties of the coating allows for controlled release of the digestive enzymes in the gastrointestinal system, preferably the region of the GI tract where the digestive enzymes are to be utilized. The coating protects the digestive enzymes from the environment and provides emulsification without detracting from the abrasion resistance of the coating. For example, for conditions requiring treatment with proteases, the release of the protease portion of the digestive enzymes is necessary in the proximal small intestine, thereby necessitating a lipid encapsulation which has a dissolution profile between 30-90 minutes. The dissolution profile may also be about 30, about 35, about 40, about 45, about 50, about 55, about 60, about 65, about 70, about 75, about 80, about 85 or about 90 minutes. Dissolution profiles may be obtained using methods and conditions known to those of skill in the art. For example, dissolution profiles can be determined at various pH's, including a pH of about 1, about 2, about 3, about 4, about 5, about 6, about 7, about 8, about 9, and about 10.

The rate of release of the digestive enzymes can also be controlled by additives as described below. When the preparations are exposed to a solvent, the solvent interacts with the mollifiable lipid in the coating and results in emulsification of the coating and release of the digestive enzymes.

The lipid to be used in the coating is any lipid, lipid mixture, or a blend of lipids and emulsifiers which emulsifies when exposed to a solvent, and has a melting point which allows the lipid to be a solid at typical storage temperatures. The lipid can be a vegetable or animal derived-lipid. In some embodiments, the lipid consists essentially of, or comprises one or more monoglycerides, diglycerides or triglycerides, or other components including, for example, emulsifiers found in hydrogenated vegetable oils. In another embodiment the lipid is a non-polar lipid.

As used herein, animal and/or vegetable “derived” lipids can include fats and oils originating from plant or animal sources and/or tissues, and/or synthetically produced based on the structures of fats and oils originating from plant or animal sources. Lipid material may be refined, extracted or purified by known chemical or mechanical processes. Certain fatty acids present in lipids, termed essential fatty acids, must be present in the mammalian diet. The lipid may, in some embodiments, comprise a Type I USP-National Formulary vegetable oil.

The coated digestive enzyme particles include, in some instances, digestive enzyme particles where about 90% of the particles are between about #40 and #140 USSS mesh in size, or from about 105 μm to about 425 μm in size. The coated digestive enzyme particles include, in some instances, digestive enzyme particles where about 75% of the particles are between about #40 and #80 USSS mesh in size, or from about 180 μm to about 425 μm in size. Particles between #40 and #140 USSS mesh in size pass through #40 mesh, but do not pass through #140 mesh.

The lipid coating reduces the aerosolization of the digestive enzymes that may be caustic to a child if inhaled through the lungs or the nose. In another embodiment, provided herein are methods of delivering coated digestive enzymes with improved safety of administration by reducing the amount of aerosolization of the digestive enzymes. The lipid coating reduces aerosolization and the potential for caustic burn, aspiration, and/or aspiration pneumonias in children and administrators of the digestive enzymes, thereby reducing the potential for illness in immune compromised children and leading to safer administration.

As used herein, the term “non-aerosolizable” will be used to refer to coated digestive enzyme particles where substantially all of the particles are large enough to eliminate or reduce aerosolization upon pouring of the coated digestive enzyme particles compared to uncoated digestive enzymes. For example, the term “non-aerosolizable” may refer to coated digestive enzyme particles where at least about 90% of the particles are between about #40 USSS mesh and #140 USSS mesh in size, or from about 106 μm to about 425 μm in size.

The term “non-aerosolizable” may also refer to coated digestive enzyme particles where at least about where at least about 75% of the particles are between about #40 and #80 mesh, or about 180 μm to 425 μm in size. The size distribution of particles in an exemplary raw enzyme preparation can vary. Large particles (>40 mesh) and very small particles (<140 mesh) are generally not suitable for proper encapsulation and can be removed by screening. Screening of the particles may include quality control steps to improve the activity, appearance or particle size of the digestive enzyme. For example, the particles may be analyzed to determine enzyme activity content, and/or visualized using chromatographic, microscopic or other analytical methods. The particles may also be screened to obtain particles of a suitable size for encapsulation by removing particles that are too fine or too large. For example, the particles may be sieved to obtain particles of a suitable size or more uniform size range for encapsulation. As a further example, the particles may be sieved through #40 USSS mesh and through USSS #140 USSS mesh. Particles that pass through the #40 USSS mesh but are retained by the #140 USSS mesh are of an appropriate size range for coating or encapsulation Particles may also be screened by sieving through USSS #140, #120, #100, #80, #70, #60, #50, #45, or #40 mesh, or any combination thereof.

In some embodiments, less than about 35, 30, 25, 20, 15 or 10% of the coated particles can be sieved through #100 USSS mesh (i.e., be about 150 μm in size). In other embodiments, less than about 20% of the coated particles can be sieved through #100 USS mesh (i.e., be about 150 μm in size). In other embodiments, or less than about 15% of the coated particles can be sieved through #100 USS mesh (i.e., be about 150 μm in size).

The amount of digestive enzymes present in the coated particles can be an amount of from about 65% to about 95% by weight. In some embodiments, the digestive enzymes can be present in the coated particles in an amount of from 70% to about 90% by weight. In other embodiments, the digestive enzymes can be present in the coated particles in an amount of from about 67.5% to about 87.5% by weight. In other embodiments, the digestive enzymes can be present in the coated particles in an amount of from about 75% to about 85% by weight. In other embodiments, the digestive enzymes can be present in the coated particles in an amount of from about 77.5% to about 82.5% by weight. In other embodiments, the amount of digestive enzymes present in the coated particles is about 65%, about 67.5%, about 70%, about 72.5%, about 75%, about 77.5%, about 80%, about 82.5%, about 85%, about 87.5%, about 90%, about 92.5%, or about 95% by weight, or anywhere in between.

In some embodiments, the pharmaceutical composition comprises at least one protease and at least one lipase, wherein the ratio of total protease to total lipase in U.S.P. units ranges from about 4:1 to about 20:1 including 4:1, 5:1, 5.371:1, 6:1, 7:1, 8:1, 9:1, 10:1, 11:1, 12:1, 13:1, 14:1, 15:1, 16:1, 17:1, 18:1, 19:1 and 20:1, along with all values in-between.

In some embodiments, the ratio of protease to lipase ranges from about 4:1 to about 10:1 including 4:1, 5:1, 5.371:1, 6:1, 7:1, 8:1, 9:1, and 10:1, along with all values in-between.

In yet another embodiment, the pharmaceutical composition comprises at least one protease and at least one lipase, wherein the ratio of total protease to total lipase in U.S.P. units/dose ranges from about 5.371:1 to about 20:1 including 5.371:1, 6:1, 7.1, 8.1, 9:1, 10:1, 11:1, 12:1, 13:1, 14:1, 15:1, 16:1, 17:1, 18:1, 19:1, and 20:1, along with all values in-between.

In another embodiment, the pharmaceutical composition comprises at least one protease and at least one lipase, wherein the ratio of total protease to total lipase in U.S.P. units/dose ranges from about 1:1 to about 20:1.

In yet another embodiment, the ratio of protease to lipase ranges from about 4:1 to about 10:1.

In one embodiment, the ratio of protease to lipase ranges from about 5.371:1 to about 10:1 including 5.371:1, 6:1, 7:1, 8:1, 9:1, and 10:1 along with all values in-between.

In one embodiment, the pharmaceutical composition comprises at least one protease and at least one amylase, wherein the ratio of total protease to total amylase in U.S.P. units/dose ranges from about 1:0.1 to about 1:10 including 1:0.25, 1:0.5, 1:0.75, 1:1, 1:1.25, 1:1.5, 1:1.75:1:2, 1:1.25, 1:1.5, 1:1.75, 1:1.2, 1:1.25, 1:1.5, 1:1.75, 1:1.2, 1:1.25, 1:1.5, 1:1.75, 1:1.2:1:1.5, 1:2, 1:3, 1:4, 1:5, 1:6, 1:7, 1:8, 1.9 and 1:10 along with all values in-between.

A pharmaceutical composition which contains the coated digestive enzyme particles can be delivered as a sprinkle, powder, capsule, tablet, pellet, caplet or other form. Allowing for specific unit dosing of coated digestive enzyme particles maintains the enzyme activity within specific stability parameters. In one non-limiting instance, the coated digestive enzyme particles are housed in a blister pack, a trilaminar foil pouch, or a similar barrier to keep out moisture and to protect the digestive enzymes from adverse environmental factors.

A pharmaceutical composition described herein can, in one instance, comprise digestive enzymes as the active ingredient. A pharmaceutical composition described herein can, in another instance, consist essentially of digestive enzymes as the active ingredient. A pharmaceutical composition described herein can, in another instance, comprise digestive enzymes as the active ingredient.

As used herein, “consisting essentially of” or “consists essentially of” means that the pharmaceutical composition does not contain one or more additional active ingredients, but may contain one or more inert additives, excipients, carriers, etc. as described in more detail below.

In one instance, one or more additives can be blended with the lipid(s) in the coating. Selection of the lipid(s) and additives can help control the rate of release of the digestive enzymes from the pharmaceutical composition. A lipid coat can be chosen to release the digestive enzymes in the area of the digestive tract to optimize treatment. In one non-limiting example, the digestive enzymes can be formulated with a lipid coating to optimize delivery to the proximal small intestines.

The pharmaceutical composition can be administered in a sachet or pouch preparation for ease of delivery to children and adults. In some embodiments, described herein are methods of administration of coated digestive enzyme particles, housed in a sachet or pouch. This facilitates administration, including but not limited to, administration in a food or a drink, via direct administration into the oral cavity, or via administration directly into the GI system through an NG-tube, G-tube or other GI entrances or deliveries.

In some embodiments, a dose of a pharmaceutical composition to be administered to a subject contains from about 100 mg to about 9000 mg (i.e., mg/dose) of the digestive enzymes. That is, each dose may contain about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2550 mg, about 2600 mg, about 2650 mg, about 2700 mg, about 2750 mg, about 2800 mg, about 2850 mg, about 2900 mg, about 2950 mg, about 3000 mg, about 3050 mg, about 3100 mg, about 3150 mg, about 3200 mg, about 3250 mg, about 3300 mg, about 3350 mg, about 3400 mg, about 3450 mg, about 3500 mg, about 3550 mg, about 3600 mg, about 3650 mg, about 3700 mg, about 3750 mg, about 3800 mg, about 3850 mg, about 3900 mg, about 3950 mg, about 4000 mg, about 4050 mg, about 4100 mg, about 4150 mg, about 4200 mg, about 4250 mg, about 4300 mg, about 4350 mg, about 4400 mg, about 4450 mg, about 4500 mg, about 4550 mg, about 4600 mg, about 4650 mg, about 4700 mg, about 4750 mg, about 4800 mg, about 4850 mg, about 4900 mg, about 4950 mg, about 5000 mg, about 5050 mg, about 5100 mg, about 5150 mg, about 5200 mg, about 5250 mg, about 5300 mg, about 5350 mg, about 5400 mg, about 5450 mg, about 5500 mg, about 5550 mg, about 5600 mg, about 5650 mg, about 5700 mg, about 5750 mg, about 5800 mg, about 5850 mg, about 5900 mg, about 5950 mg, about 6000 mg, about 6050 mg, about 6100 mg, about 6150 mg, about 6200 mg, about 6250 mg, about 6300 mg, about 6350 mg, about 6400 mg, about 6450 mg, about 6500 mg, about 6550 mg, about 6600 mg, about 6650 mg, about 6700 mg, about 6750 mg, about 6800 mg, about 6850 mg, about 6900 mg, about 6950 mg, about 7000 mg, about 7050 mg, about 7100 mg, about 7150 mg, about 7200 mg, about 7250 mg, about 7300 mg, about 7350 mg, about 7400 mg, about 7450 mg, about 7500 mg, about 7550 mg, about 7600 mg, about 7650 mg, about 7700 mg, about 7750 mg, about 7800 mg, about 7850 mg, about 7900 mg, about 7950 mg, about 8000 mg, about 8050 mg, about 8100 mg, about 8150 mg, about 8200 mg, about 8250 mg, about 8300 mg, about 8350 mg, about 8400 mg, about 8450 mg, about 8500 mg, about 8550 mg, about 8600 mg, about 8650 mg, about 8700 mg, about 8750 mg, about 8800 mg, about 8850 mg, about 8900 mg, about 8950 mg, or about 9000 mg of the digestive enzymes.

In one non-limiting example, a pharmaceutical composition comprises coated digestive enzyme particles that are encapsulated, and the capsule comprises about 900 mg of the digestive enzymes. A subject may be administered 3, 4, 5, 6, 7, 8, 9, 10, 11 or 12 capsules at one time depending upon the age, weight, severity of the addiction, and/or general health of the subject.

For explanation purposes only, in one non-limiting example, a subject can be administered 5 capsules with breakfast and each capsule comprises about 900 mg of digestive enzymes. In such an administration, the subject will be administered about 4,500 mg of digestive enzymes.

The protease activity of a dose of a pharmaceutical composition described herein can be an amount such as, for example, not less than about 100 U.S.P. Units/mg, about 105 U.S.P. Units/mg, about 110 U.S.P. Units/mg, about 115 U.S.P. Units/mg, about 120 U.S.P. Units/mg, about 125 U.S.P. Units/mg, about 130 U.S.P. Units/mg, about 135 U.S.P. Units/mg, about 140 U.S.P. Units/mg, about 145 U.S.P. Units/mg, about 150 U.S.P. Units/mg, about 155 U.S.P. Units/mg, about 160 U.S.P. Units/mg, about 165 U.S.P. Units/mg, about 170 U.S.P. Units/mg, about 175 U.S.P. Units/mg, about 180 U.S.P. Units/mg, about 185 U.S.P. Units/mg, about 190 U.S.P. Units/mg, about 195 U.S.P. Units/mg, or about 200 U.S.P. Units/mg.

In one instance, the amylase is present in the pharmaceutical composition in an amount of from about 120,000 to about 370,000 U.S.P. units/unit dose.

In one instance, the protease is present in the pharmaceutical composition in an amount of from about 130,000 to about 165,000 U.S.P. units/unit dose.

In one instance, the lipase is present in the pharmaceutical composition in an amount of from about 17,000 to about 60,000 U.S.P. units/unit dose.

For explanation purposes only, in one non-limiting example, a subject can be administered 8 capsules with lunch and each capsule comprises about digestive enzymes having a protease activity of from about 130,000 U.S.P. Units to about 150,000 U.S.P. Units of protease activity. In such an administration, the subject will be administered about 4,500 mg of digestive enzymes.

The term “unit dose” when used in reference to a pharmaceutical composition refers to physically distinct units suitable as unitary dosage for subjects, each unit containing a predetermined quantity of active material calculated to produce the desired therapeutic effect in association with the required diluent; i.e., carrier, or vehicle.

In some instances, one, two, three, four, five, six, seven, eight, nine, or more doses or more of a pharmaceutical composition can be administered to a subject daily.

In other instances, a subject may be administered one or more doses of a pharmaceutical composition once, twice, three times, four times, or more daily.

In one non-limiting example, a subject is administered from about 4 to about 10 capsules with breakfast, from about 4 to about 10 capsules with lunch and from about 4 to about 10 capsules with dinner.

A practitioner can empirically determine the dose of the pharmaceutical composition to be administered to a subject depending upon, for example, the height, weight, age, severity of addiction, length of addiction, etc. A subject may receive treatment for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 weeks, about 26 weeks, about 27 weeks, about 28 weeks, about 29 weeks, about 30 weeks or more weeks. A subject may receive treatment for about 5 days, about 10 days, about 15 days, about 20 days, about 25 days, 30 days, about 35 days, about 40 days, about 45 days, 50 days, about 55 days, about 60 days, about 65 days, 70 days, about 75 days, about 80 days, about 85 days, about 90 days, about 95 days, about 100 days, about 105 days or more. It will be understood that a subject having received treatment and no longer showing or feeling one or more signs of the addiction can be administered one or more treatments if relapse occurs.

It will be understood that if a subject is highly addicted to one or more substances, the subject can be administered higher doses of the pharmaceutical composition. Alternatively, if a subject is only mildly addicted to one or more substances, the subject can be administered lower doses of the pharmaceutical composition. A subject may receive increased or decreased doses of the pharmaceutical composition as throughout their treatment depending upon how they react to the pharmaceutical composition.

In some embodiments, the invention relates to the administration of coated digestive enzyme particles, housed in a sachet which allows for particular types of administration including but not limited to administration in food, drink, or direct administration into the oral cavity or directly into the GI system through a NG-tube, G-tube or other GI entrances. The sachet can represent a single unit dose or a multiple unit dose. The trilaminar foil allows the coated digestive enzyme particles to remain stable during storage and allows for ease of administration.

In other embodiments, the invention relates to coated digestive enzyme particles that are encapsulated and stored in moisture resistant blister packs or foil-sealed moisture resistant bottles. Appropriate foil-sealed moisture resistant bottles are known in the art and include, for example, glass bottles, high density polyethylene (HTPE) plastic bottles or other appropriate type of glass and plastic bottles that can be sealed with foil. In one non-limiting example, capsules are stored in a sachet. Pharmaceutical compositions described herein are packaged, in certain instances, under low humidity conditions. As used herein, “low humidity conditions” refer to packaging in less than or equal to 15% relative humidity. It would be understood that an appropriate pharmaceutical-grade desiccant can also be packaged, in some instances, with the product to extend shelf life during storage.

Pharmaceutical compositions described herein, in some instances, can include not only one or more digestive enzymes, but also one or more carriers, one or more excipients, one or more buffers, one or more fillers, one or more binders, one or more preservatives, one or more anti-oxidants, one or more alkyl parabens, one or more proteins, one or more sugars, one or more chelating agents, one or more sugars, one or more surfactants, one or more salt-forming counter ions, one or more stabilizers, one or more surfactants, one or more diluents, one or more sweeteners, one or more salts, one or more taste maskers, etc. Acceptable carriers, excipients, or stabilizers are those that are non-toxic to recipients at the dosages and concentrations employed, and include buffers such as phosphate, histidine, acetate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin or gelatin; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® and/or polyethylene glycol (PEG). In some instances, the coated digestive enzyme particles are prepared in microcrystalline cellulose.

Acceptable carriers are physiologically acceptable to the administered subject and retain the therapeutic properties of the compounds with/in which it is administered. Acceptable carriers and their formulations are and generally described in, for example, Remington' pharmaceutical Sciences (18th Edition, Ed., A. Gennaro, Mack Publishing Co., Easton, PA 1990). One exemplary carrier is physiological saline.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier is acceptable in the sense of being compatible with the other ingredients of the formulation and not injurious to a subject to whom it is administered. Nor should an acceptable carrier alter the specific activity of the active agents in the pharmaceutical composition.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with administration to a human subject.

Another embodiment of the invention relates to the improvement of delivery of enzymes to humans by reducing the use of excipients, extenders and solvents currently used in the preparations for delivery of digestive enzymes to humans. For example, preparation pharmaceutical composition may contain only one excipient, which increases the safety of administration by decreasing the chance of an allergic response.

In some embodiments, the lipid coating does not require the digestive enzymes to be treated with solvents, extenders and excipients to facilitate flow or improve stability of the coated particles. In one aspect, provided herein is a “clean” preparation of GRAS substances (generally regarded as safe) to be administered. The reduction in the use of solvents, extenders excipients and other additives reduces the exposure of the subjects taking the pharmaceutical compositions to potential allergens, thereby producing a hypoallergenic composition that further enhances its potential uses in the treatment of subjects who might otherwise develop an allergic response to treatment. Administration of the coated digestive enzyme particles described herein can, thus, reduce exposure to potentially toxic substances and will also reduce the possibility of allergy formation. Accordingly, in some embodiments, the pharmaceutical compositions described herein are hypoallergenic.

Coated digestive enzyme particles supplied by an API supplier may be provided as irregular shaped, and multi-sized particles, with uneven edges, and much clumping, and containing some crystalline salt particles. Uneven particle size and shape reduces flow properties, and interferes with packaging. Pouring uncoated enzyme into the mouth of an individual would be difficult, and potentially may cause too much or too little of the enzyme to be delivered. The coated digestive enzyme particles described herein provide a non-dusty, free-flowing particulate preparation suitable for sachet packaging and for pouring onto food or drink. In addition, the coating helps prevent aerosolization, and therefore increase safety, and to increase flow properties which enhance manufacturing of a pharmaceutical composition to be administered in the described methods.

The lipid can be present in the coated particles in an amount of about 15%, about 16%, about 16.5%, about 17%, about 17.5%, about 18%, about 18.5%, about 19%, about 19.5%, about 20%, about 20.5%, about 21%, about 21.5%, about 22%, about 22.5%, about 23%, about 24% or about 25% by weight.

“Lipids” as used herein means those lipids which contain at least one hydrophilic group and at least one hydrophobic group, and have a structure capable of forming a hydrophilic and hydrophobic interface. These chemical and/or physical properties, mentioned above, of a lipid permit emulsification. Examples of interfaces include, for example, micelles and bilayers. The hydrophilic group can be a polar group and can be charged or uncharged.

The lipid can be derived from animal origins or vegetable origins, such as, for example, palm kernel oil, soybean oil, cottonseed oil, canola oil, and poultry fat, including hydrogenated type I vegetable oils. In some embodiments, the lipid is hydrogenated. The lipid can also be saturated or partially saturated. Examples of lipids include, but are not limited to, monoglycerides, diglycerides, triglycerides, fatty acids, esters of fatty acids, phospholipids, salts thereof, and combinations thereof. In one non-limiting instance, the lipid coating comprises monoglycerides, diglycerides, triglycerides, or a combination thereof. In another non-limiting instance, the lipid coating comprises a combination of monoglycerides and diglycerides. In another non-limiting instance, the lipid coating comprises a combination of diglycerides and triglycerides. In another non-limiting instance, the lipid coating comprises a combination of monoglycerides, diglycerides and triglycerides.

The lipid may be, in some instances, a “food grade lipid”. Examples of food grade lipids include, but are not limited to, sorbitan monostearates, sorbitan tristearates, and calcium stearoyl lactylates. Other examples of food grade fatty acid esters which are lipids include acetic acid esters of mono- and diglycerides, citric acid esters of mono- and di-glycerides, lactic acid esters of mono- and di-glycerides, polyglycerol esters of fatty acids, propylene glycol esters of fatty acids, and diacetyl tartaric acid esters of mono- and diglycerides. In one non-limiting example, a lipid coating comprises a hydrogenated soy oil.

The lipid may also be, in some instances, a pharmaceutical grade lipid. Pharmaceutical grade lipids include, but are not limited to, highly purified lipid from which all protein antigens have been removed. Such lipids are beneficial in that they do not induce allergic responses and do not include cis or trans fatty acids. One non-limiting example of a pharmaceutical grade lipid comprises a soybean oil that is fully hydrogenated.

In some instances, a lipid coating will produce non-agglomerating, non-aerosolizing digestive enzyme particles.

The inclusion of one or more additives with a lipid can be used to control emulsification or dissolution of the coating and release of the digestive enzymes. For example, a triglyceride, can be blended with a monoglycerides to control emulsification or dissolution of the coating and thus control (e.g., decrease) the rate of release of the digestive enzymes from the coated particles. As a further example, a diglyceride and a triglyceride can be blended with a monoglyceride to control the rate of release of the digestive enzymes. Hydrogenated vegetable oils may contain emulsifying agents, such as soy lecithin or other components.

Properties including mechanical strength, melting point, and hydrophobicity can be considered when choosing a suitable lipid coating for the digestive enzymes. Lipids having lower melting points or more polar, hydrophilic properties are generally less suitable for the coating because they may result in a product that would cake under accelerated storage stability conditions. Coated digestive enzyme particles made using, for example, hydrogenated soy oil (e.g., partially or fully hydrogenated), hydrogenated castor wax, and carnauba wax all demonstrated good pouring and no caking.

The wax can be paraffin wax; a petroleum wax; a mineral wax such as ozokerite, ceresin, or montan wax; a vegetable wax such as, for example, carnauba wax, bayberry wax or flax wax; an animal wax such as, for example, spermaceti; or an insect wax such as beeswax.

Additionally, the wax material can be an ester of a fatty acid having 12 to 31 carbon atoms and a fatty alcohol having 12 to 31 carbon atoms, the ester having from a carbon atom content of from 24 to 62, or a mixture thereof Examples include myricyl palmitate, cetyl palmitate, myricyl cerotate, cetyl myristate, ceryl palmitate, ceryl certate, myricyl melissate, stearyl palmitate, stearyl myristate, and lauryl laurate. [00198]“Encapsulate” as used herein, means that the coated digestive enzyme particles are packaged in an appropriate capsule material. Appropriate capsule materials are known in the art and are contemplated for use herein.

A non-limiting exemplary dosage form is a capsule in which the coated digestive enzyme particles are packaged in an appropriate material to be delivered as a capsule dosage form. A capsule, as described herein, includes soft or hard capsules, e.g., capsules made from animal-derived gelatin or plant-derived Hydroxypropylmethyl cellulose (HPMC), or “sprinkle capsules”).

Exemplary capsules include, but are not limited to, HPMC capsules from CAPSUGEL®. As used herein, hydroxypropyl methylcellulose includes, but is not limited to, HPMC K100, HPMC K4M, HPMC K15M, and HPMC K100M.

In some embodiments, a dosage form comprises a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) solid dispersion, solid solution, bioerodible dosage form, controlled release formulations, pulsatile release dosage forms, multiparticulate dosage forms. In other embodiments, the pharmaceutical formulation is in the form of a powder. In still other embodiments, the pharmaceutical formulation is in the form of a tablet, including but not limited to, a fast-melt tablet.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In one non-limiting embodiment, a capsule may be prepared, for example, by placing the composition described above, inside of a capsule. In some embodiments, the composition is placed in a soft gelatin capsule. In other embodiments, the composition is placed in standard gelatin capsules or non-gelatin capsules such as capsules comprising HPMC. In other embodiments, the composition is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the composition is delivered in a capsule form.

A non-limiting exemplary route of administration comprises oral administration of a composition described herein. Pharmaceutical compositions can be administered as a single capsule or in multiple capsule dosage form. In some embodiments, the pharmaceutical formulation is administered in two, or three, four, five, six, seven, eight, nine, ten, eleven, twelve, thirteen or fourteen capsules or tablets at each time of administration.

One non-limiting exemplary dosing regimen includes, for example, administration of from about 4 to about 9 capsules, or from about 5 to about 8 capsules with each meal daily for at least 12 weeks. Another non-limiting exemplary dosing regimen includes, for example, administration of from about 4 to about 9 capsules, or from about 5 to about 8 capsules with each meal daily for at least 24 weeks. Another non-limiting exemplary dosing regimen includes, for example, administration of from about 4 to about 9 capsules, or from about 5 to about 8 capsules with each meal daily for at least 30 weeks.

A composition described herein includes, in one instance, a lipid-encapsulated pancreatic enzyme concentrate (PEC) that comprises one or more proteases, one or more lipases and one or more amylases, designed to release chymotrypsin and other proteases in the proximal small intestine without extreme degradation by stomach acid.

In one instance, the composition comprises a protease, wherein the protease comprises chymotrypsin. Chymotrypsin is a serine protease enzyme and one of the prominent proteases involved in human digestion. Emerging evidence demonstrates that amino acids, which are the by-product of protein digestion, are not only involved in protein synthesis but also play a significant role in other important neurological functions. Of those essential amino acids cleaved by chymotrypsin, phenylalanine and tryptophan are necessary components of the body's synthesis of dopamine and serotonin respectively. Chymotrypsin is unique because it cleaves proteins at the sites proximal to four essential amino acids. See, FIG. 1.

There is an emergence of evidence with respect to cocaine addiction and the role of ΔFosβ that builds up in the brains of cocaine addicts that causes an elongation of and proliferation of dendritic spines. These dendritic spines may be associated with the persistence of cravings in subjects that are addicted to one or more substances.

Compositions as described herein cleave phenylalanine from endogenous proteins that are ingested. Its administration may hasten the buildup of phenylalanine and hence replenish the depleted dopamine stores. This allows for the synthesis of new dopamine. Further, the presence of additional phenylalanine may drive the reduction of the elongated dendritic spines, and therefore reduce the cravings for cocaine, or another drug or alcohol.

A composition described herein a high protease, lipid-encapsulated, pancreatic enzyme concentrate (PEC) designed to release chymotrypsin and other proteases in the proximal small intestine without extreme degradation by stomach acid. It is a formulation that allows high protease delivery without a classic enteric coating and is designed to deliver enzyme to the proximal small intestine (duodenum). This formulation allows for maximal delivery of proteases into the proximal small intestines where protease delivery and subsequent protein hydrolysis and peptide absorption is best optimized.

The pharmaceutical compositions described herein are suited for use in delivering digestive enzymes to subjects having one or more symptoms of addiction to one or more substances. The pharmaceutical compositions described herein are suited for use in delivering digestive enzymes to treat a subject addicted to one or more substances.

Treating”, as used herein, refers to partial or complete reduction of one or more symptoms of addiction to one or more substances. In some instances, treatment can be assessed using conventional means in the art for measuring the severity of a symptom, for example, how strong a craving is for an addicted substance, other symptoms. In another embodiment, treatment can be assessed by quantitatively measuring a level of ΔFosβ from a sample obtained from a subject and comparing the level of ΔFosβ in the sample to a level of ΔFosβ from a healthy (non-addicted) subject. Methods for measuring ΔFosβ include, but are not limited to, an Enzyme Linked Immunosorbant Assay (ELISA).

### Pancreatin and Pancrealipase

There are two types of pancreatic enzymes which have United States Pharmacopeia (U.S.P.) designations: pancreatin and pancrealipase. “Pancreatin” is a substance containing enzymes, principally amylase, lipase, and protease, obtained from the pancreas of the hog Sus scrofa Linne var. domesticus Gray (Fam. Suidae) or of the ox Bos Taurus Linne (Fam. Bocidae). Pancreatin contains, in each mg, not less than 25 U.S.P. units of amylase activity, not less than 2 U.S.P. units of lipase activity, and not less than 25 U.S.P. of protease activity. Pancreatin of a higher digestive power may be labeled as a whole-number multiple of the three minimum activities or may be diluted by admixture with lactose, or with sucrose containing not more than 3.25 percent of starch, or with pancreatin of a lower digestive power. Pancreatin can be provided as a crystalline substance.

In contrast, pancrealipase refers to a cream-colored, amorphous powder, having a faint, characteristic meaty odor, which contains lipase in an amount of not less than 24 U.S.P. Units/mg; protease in an amount of not less than 100 U.S.P. Units/mg; and amylase in an amount of not less than 100 U.S.P. Units/mg; with not more than 5% fat and not more than 5% loss on drying. CREON® is a form of pancrealipase that is sold as formulations of (i) 3,000 Units of a lipase, 9,500 Units of a protease, 15,000 Units of an amylase; (ii) 6,000 Units of a lipase, 19,000 Units of a protease, 30,000 Units of an amylase; (iii) 12,000 Units of a lipase, 38,000 Units of a protease, 60,000 Units of an amylase; (iv) 24,000 Units of a lipase, 76,000 Units of a protease, and 120,000 Units of an amylase; or (v) 36,000 Units of a lipase, 114,000 Units of a protease, and 180,000 Units of an amylase. CREON® formulations are known to be irritating to mucosa of a subject and also is known to cause the following adverse side effects: Abdominal pain, abnormal feces, cough, dizziness, flatulence, headache, weight decreased; hyperuricemia, fibrosing colonopathy (with high doses), and/or allergic reactions.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1

### Proposed Indication

The compositions and method described are for treatment of an addiction. For example, administration of the pharmaceutical composition can treat a craving, impulsivity, lack of impulse control, or a combination thereof, of the addiction. The present example describes treatment of cravings, impulsivity, and lack of impulse control in active cocaine-addicted users.

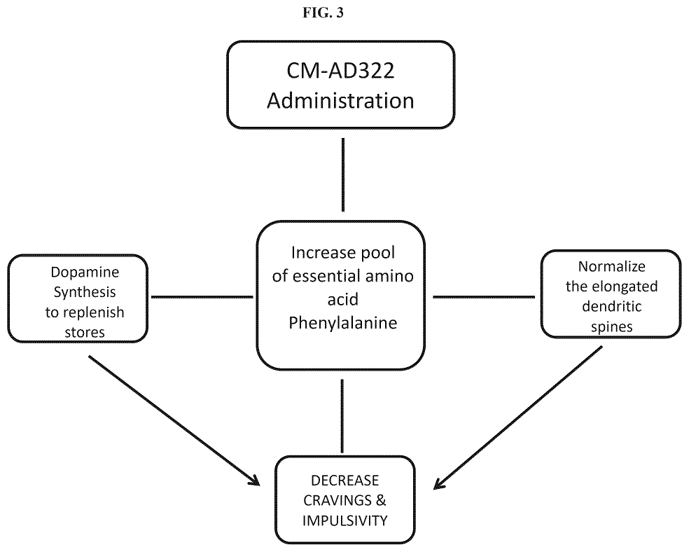

FIG. 3 illustrates the synthesis of thinking on the potential effectiveness of CM-AD322.

Due to ΔFosβ buildup and subsequent reinforcement of addictive behaviors, the effectiveness of CM-AD332 on cocaine cravings in active cocaine addicts is examined.

Standard measures of cravings and the addictive behaviors, as well as brain changes that occur as a result of the administration of CM-AD322 are assessed.

### Dosage Form, Route of Administration, and Dosing Regimen

CM-AD322 is a formulation that comprises coated digestive enzyme particles, which particles contain a core comprising digestive enzymes and a lipid coating, where the digestive enzymes are present in the coated particles in an amount of from about 70% to about 90%. The digestive enzymes comprise a mixture of a protease, an amylase and a lipase. The coated digestive enzyme particles are encapsulated for administration to a subject as capsules. CM-AD322 delivers a high dose of protease to the subject in the amount of from about 135,000 to about 150,000 USP units of protease per capsule.

CM-AD322 is designed to release chymotrypsin and other proteases such as trypsin in the proximal small intestine without extreme degradation by stomach acid.

Chymotrypsin is a protease enzyme in the mixture of enzymes. It is a serine protease enzyme and one of the prominent proteases involved in human digestion. Chymotrypsin is unique because it cleaves proteins at the sites proximal to four essential amino acids. See, FIG. 1.

Due to ΔFosβ buildup and subsequent reinforcement of addictive behaviors, the effectiveness of CM-AD332 on cocaine cravings in active cocaine addicts is assessed.

Standard measures of cravings and the addictive behaviors, as well as brain changes that occur as a result of the administration of CM-AD322 are assessed.

CM-AD322 for treatment of cocaine addiction comprises coated digestive enzyme particles in capsule form.

### Packaging

The capsule compositions can be packaged in blister packs with foil backing for size 00 or 0 capsules, dependent on fill characteristics.

Clinical supplies are manufactured under cGCP conditions.

### Clinical Trial

The present example describes treatment of cocaine addiction in human subjects that are from about 16 to about 70 years old.

The trial design as presented may include active addicts as studied in the following treatment arms:

| Group A: Addicted | Group B: Addicted | Group C: Addicted |
|---|---|---|
| Dosage A | Dosage B | Placebo |

The trial design as presented may also include one group of non-addicted subjects as shown in the following treatment arms:

| Group A | Group B | Group C | Group D |
|---|---|---|---|
| Addicted | Addicted | Addicted | Non-addicted |
| Dosage A | Dosage B | Placebo | Placebo |

A placebo to be administered to the subjects of Groups C and/or D include compositions that do not comprise the digestive enzymes.

Subjects in the study are administered 5-8 capsules with each meal, three times daily, for at least 12 weeks. A subject will receive approximately 360,000 U.S.P. Units/per dose.

### Primary Outcome Measures

A primary outcome measure to be tested is cocaine craving as measured by a cocaine craving questionnaire.

The cocaine craving questionnaire (CCQ) is a 45-item self-report questionnaire that assesses cocaine craving. The CCQ is an art-recognized method of assessing addiction cravings. The CCQ-Now version asks about current craving for cocaine, and the CCQ-general version asks about average craving over the preceding week Items for the questionnaire were generated to represent five distinct conceptualizations of cocaine craving: (1) desire to use cocaine; (2) anticipation of positive outcomes from cocaine use; (3) anticipation of relief from cocaine withdrawal symptoms or relief from negative mood; (4) intention and planning to use cocaine; (5) lack of control over use. The first 4 item categories were adapted from the QSU.

### Secondary Outcome Measures

Secondary outcome measures that are to be measured are, for example, impulsivity and impulse control as measured by the Barratt Impulsiveness Scale. The Barratt Impulsiveness Scale (BIS-11; Patton et al., 1995, J. Clin. Psy., vol. 51, pp. 768-774) is an art-recognized questionnaire designed to assess the personality/behavioral construct of impulsiveness and to measure some of the ways in which a subject acts and thinks.

People differ in the ways they act and think in different situations; items are scored on a 4-point scale: Rarely/Never=1; Occasionally=2; Often=3; or Almost Always/Always=4.

| | |
|---|---|
| 1 | I plan tasks carefully. |
| 2 | I do things without thinking. |
| 3 | I make-up my mind quickly. |
| 4 | I am happy-go-lucky. |
| 5 | I don't “pay attention.” |
| 6 | I have “racing” thoughts. |
| 7 | I plan trips well ahead of time. |
| 8 | I am self-controlled. |
| 9 | I concentrate easily. |
| 10 | I save regularly. |
| 11 | I “squirm” at plays or lectures. |
| 12 | I am a careful thinker. |
| 13 | I plan for job security. |
| 14 | I say things without thinking. |
| 15 | I like to think about complex problems. |
| 16 | I change jobs. |
| 17 | I act “on impulse.” |
| 18 | I get easily bored when solving thought problems. |
| 19 | I act on the spur of the moment. |
| 20 | I am a steady thinker. |
| 21 | I change residences. |
| 22 | I buy things on impulse. |
| 23 | I can only think about one thing at a time. |
| 24 | I change hobbies. |
| 25 | I spend or charge more than I earn. |
| 26 | I often have extraneous thoughts when thinking. |
| 27 | I am more interested in the present than the future. |
| 28 | I am restless at the theater or lectures. |
| 29 | I like puzzles. |
| 30 | I am future oriented. |

### Other Outcome Measures

This Phase II trial includes the following non-safety, non-functional MRI, exploratory outcome measures: Timeline Follow Back (TLFB); World Health Organization Quality of Life Questionnaire (WHOQOL-BREF); Hamilton Rating Scale for Depression (HAM-D); Urine Toxicology; Blood Phenylalanine/Leucine Ratio; Fecal Chymotrypsin; Body Mass Index; Initiation of Abstinence; and/or Length of Abstinence.

### Neuroimaging Outcome Measures

A Phase II clinical trial also includes the following Exploratory Neuroimaging Outcome Measures during which the following will be assessed:

Tasks: Go/No-Go Response Inhibition Task; and/or Drug Cue Caving Task.

During this imaging test: Functional Magnetic Resonance Imaging (fMRI).

In addition, the following neuroimaging studies will be conducted: Diffusion Tensor Imaging (DTI) and/or Structural Magnetic Resonance Imaging (sMRI).

### Example 2

Subjects are treated according to the methods of Example 1, and the level of phenylalanine in blood is measured prior to commencement of treatment, and at one or more times during treatment.

The blood level of phenylalanine in of addicted patients administered CM-AD322 or placebo are compared at each time point to a normal level of phenylalanine in healthy subjects (i.e., from about 2 mg/dL to about 6 mg/dL, or from about 120 micromolar to about 360 micromolar). Depending upon the blood level of phenylalanine, a practitioner can determine how to modify the treatment plan of the subject being treated, or to continue with the same treatment plan.

Blood levels of phenylalanine can also be measured at one or more time points after the subject has stopped treatment with CM-AD322 to determine if the subject should receive additional treatment.

### Example 3

Context-induced reinstatement rodent models are utilized to assess the effect of digestive enzyme compositions described herein.

Laboratory animals (generally, Fisher 344 rats or Sprague-Dawley rats) are first trained to self-administer cocaine in an environment (termed context A) associated with a specific set of “background” stimuli (e.g., operant conditioning chamber fan, time of day, visual cues, tactile cues). Lever pressing is then extinguished in a different environment (termed context B) with a different set of “background” stimuli. During reinstatement testing under extinction conditions, exposure to context A previously paired with cocaine reinstates lever responding. In such a rodent model, there are two arms of the study: (1) animals which are administered cocaine and a placebo and (2) antibodies which are administered cocaine and a composition described herein. Each arm will include 4-6 rats. Methods of delivering cocaine to rodents in their water bottles are known in the art. Compositions as described herein can be administered with food. The procedure is based on a “renewal” procedure that has been used to assess the role of contexts in resumption of conditioned responses to aversive and appetitive cues after extinction. Responses in each arm are compared and statistical significance is determined.

### Example 4

Discrete cues-induced reinstatement models are utilized to assess the effect of digestive enzyme compositions described herein.

Laboratory animals (generally, Fisher 344 rats or Sprague-Dawley rats) are first trained to self-administer cocaine; cocaine delivery is temporally paired with a discrete cue (e.g., tone, light, etc.). Lever pressing is then extinguished in the absence of cocaine and the cue. During reinstatement testing, exposure to the discrete cue, which is earned contingently during testing, reinstates lever responding. In such a rodent model, there are two arms of the study: (1) animals which are administered cocaine and a placebo and (2) antibodies which are administered cocaine and a composition described herein. Each arm will include 4-6 rats. Methods of delivering cocaine to rodents in their water bottles are known in the art. Compositions as described herein can be administered with food. Responses in each arm are compared and statistical significance is determined.

### Example 5

Discriminative cues-induced reinstatement models are utilized to assess the effect of digestive enzyme compositions described herein.

Laboratory animals (generally, Fisher 344 rats or Sprague-Dawley rats) are trained to self-administer cocaine in the presence of distinct discriminative stimuli (e.g., visual cues, olfactory cues); one set of stimuli signals cocaine availability (S+) and the other signals unavailability (S−). Lever pressing is then extinguished in the absence of the discriminative stimuli and cocaine. During the reinstatement test, re-exposure to the S+, but not S−, reinstates operant conditioned responding. In such a rodent model, there are two arms of the study: (1) animals which are administered cocaine and a placebo and (2) antibodies which are administered cocaine and a composition described herein. Each arm will include 4-6 rats. Methods of delivering cocaine to rodents in their water bottles are known in the art. Compositions as described herein can be administered with food. Responses in each arm are compared and statistical significance is determined.

### Example 6

Drug-priming-induced reinstatement models are utilized to assess the effect of digestive enzyme compositions described herein.

Laboratory animals (generally, Fisher 344 rats or Sprague-Dawley rats) are first trained to self-administer cocaine; typically cocaine delivery is paired with a discrete cue. Lever pressing is then extinguished in the presence of the discrete cue. During reinstatement testing under extinction conditions (usually, in the presence of the discrete cue), pre-session non-contingent priming injections of the previously self-administered cocaine or related cocaine reinstate lever responding. In such a rodent model, there are two arms of the study: (1) animals which are administered the drug and a placebo and (2) antibodies which are administered the drug and a composition described herein. Each arm will include 4-6 rats. Methods of delivering cocaine to rodents in their water bottles are known in the art. Compositions as described herein can be administered with food. Responses in each arm are compared and statistical significance is determined.

### Example 7

Reinstatement models are utilized to assess the effect of digestive enzyme compositions described herein.

A reinstatement model is an animal model of cocaine relapse in which laboratory animals are tested for reinstatement of cocaine seeking induced by cocaine-priming, discrete cues, discriminative cues, contextual cues, and/or stressors, following cocaine self-administration training (typically lever-press or nose-poke for drug infusions) and subsequent extinction of the cocaine-reinforced responding. Less frequently used procedural variations of the reinstatement model include, for example, operant-conditioning-based runway and Pavlovian-conditioning-based conditioned place preference procedures. In such a rodent model, there are two arms of the study: (1) animals which are administered cocaine and a placebo and (2) antibodies which are administered cocaine and a composition described herein. Typically, Fisher 344 rats or Sprague-Dawley rats are utilized; each arm will include 4-6 rats. Methods of delivering cocaine to rodents in their water bottles are known in the art. Compositions as described herein can be administered with food. Responses in each arm are compared and statistical significance is determined.

### Example 8

Stress-induced reinstatement models are utilized to assess the effect of digestive enzyme compositions described herein.

Laboratory animals (generally, Fisher 344 rats or Sprague-Dawley rats) are first trained to self-administer cocaine; cocaine delivery is temporally paired with a discrete cue. Lever pressing is then extinguished in the presence of the discrete cue. During reinstatement testing under extinction conditions (in the presence of the discrete cue), pre-session exposure to stressors (typically intermittent footshock or yohimibine injection) reinstates lever responding. In such a rodent model, there are two arms of the study: (1) animals which are administered cocaine and a placebo and (2) antibodies which are administered cocaine and a composition described herein. Each arm will include 4-6 rats. Methods of delivering cocaine to rodents in their water bottles are known in the art. Compositions as described herein can be administered with food. Responses in each arm are compared and statistical significance is determined.

### Example 9

This example describes a multi-staged procedure, which started with the acquisition of cocaine IVSA, followed by several cycles of extinction and reinstatement.

Male C57BL/6J mice are obtained at the age of 8-9 weeks and housed four per cage with food and water ad libitum.

Each operant chamber is fitted with an AL (active lever) and an IAL (inactive lever), a cue light is positioned 45 mm above the center of the two levers, and a house light is positioned on the opposite wall of the chamber. The position of the AL is counterbalanced across individuals and within each group. Chambers are located in sound-attenuating containers with a fan that always is on during training sessions.

Mice are anesthetized and implanted with a chronic indwelling catheter.

One week after jugular catheter implantation, training of mice is initiated. The training sessions last 2 h/day, 5 days a week for 3-4 weeks. Each session starts with the house light on and both levers extended. For master mice, the training starts with an FR 1 (fixed ratio 1) schedule of reinforcement, during which one response to the assigned AL results in the programmed consequences, i.e., an IV drug injection through the pre-implanted catheter as well as illumination of the central cue light on and the extinguishing of the house light for 20 s (time-out, no more is was delivered after active response).

The criteria for moving from FR1 to FR5 phase includes: (1) earning a minimum of 20 reinforcers per session in two consecutive sessions; (2) varying in the number of infusions earned by no more than 20% in two consecutive sessions; (3) making at least 70% of all responses on the AL; and/or (4) the passage of at least 2 weeks in the FR1 phase. The FR5 training phase usually lasts 1-2 weeks and progresses to the extinction phase when no more than 20% variation in the number of reinforcers earned between the two sessions occurs.

Mice in a saline control group are trained with the same procedure except the cocaine solution is replaced with saline.

Mice in a treatment test group are trained with the same procedure except food is supplemented with coated digestive enzyme particles.

The training procedure of mice in the yoked group strictly follows that of the corresponding paired masters, except each drug delivery and presentation of visual cues are initiated by the paired masters.

Mice in all groups are trained in operant chambers with everything similar as in the acquisition phase except that all lever presses are recorded, but do not include programmed consequences. Subjects remain in extinction until no more than 20% variation in the number of AL responses between two consecutive sessions occurs, with a minimum of 2-week extinction period.

Four cycles of reinstatement testing are performed in all subjects 24 h after meeting the extinction criteria. In each cycle, mice are tested for their propensity to reinstate drug-seeking behavior after a challenge injection of cocaine (0, 1, 3.2 and 10 mg/kg IP, in random order) followed by at least 2-day re-extinction until they met again the extinction criteria. The in vitro electrophysiological studies are performed 24 h after the last extinction session. The end point of behavioral training occurs when the subjects are at the extinction phase.

Data are assessed using analysis of variance (ANOVA). Post-hoc analyses are conducted using Tukey tests and analyses are considered statistically significant at P<0.05. All data are presented as mean±SEM. Correlations are generated using Pearson's product correlations to test for correlations of responses between the different reinstatement tests.

### Example 10

Heroin addiction and treatment can be assessed using an art-recognized animal model. Male, Sprague-Dawley rats are individually housed and are given water ad libitum and are maintained on 25 g of standard rat chow per day for the duration of each experiment. Rats are acclimated to handling and allowed to adapt for a minimum of four days prior to the start of the experiment.

Rats are trained to lever press in standard self-administration chambers linked to a computerized data collection program (MED-PC, Med Associates, Inc., St. Albans, VT, USA). The chambers are equipped with two retractable levers, a white stimulus light above each lever, a food pellet dispenser between the levers, a tone generator, and a house light on the wall opposite the levers. Each chamber contains a sound-attenuating cubicle equipped with a ventilation fan. Rats are food deprived overnight and trained to lever press on a fixed ratio 1 (FR1) schedule of food reinforcement during a 15-h overnight training session in the absence of explicit conditioned stimulus (CS) presentation (i.e., active lever presses resulted in the delivery of a food pellet only). Lever presses on an inactive lever are recorded. Following lever response training, food dispensers are permanently removed from the test chambers.

Rats are administered a placebo (control arm) or food containing coated digestive enzyme particles (test arm) with food.

Rats begin self-administration of heroin (diacetylmorphine HCl,) along a FR1 schedule at an initial dose of 50 μg/50 l/infusion for 2 days, followed by 10-12 days of self-administration at a dose of 25 g/50 μl/infusion. At the start of each 3-h session, the catheter is connected to a liquid swivel (Instech, Plymouth Meeting, PA) via polyethylene 20 tubing that was encased in steel spring leashes (Plastics One, Inc.). The swivels are suspended above the operant conditioning chamber and are connected to infusion pumps (model PHM-100, Med-Associates). The house light signals the initiation of the session and remains illuminated throughout the entire session. Active lever responses result in a 2 s activation of the infusion pump and a 5 s presentation of a conditioned stimulus complex (CS), which includes a cue light above the active lever and a tone (78 dB, 4.5 kHz). Following each infusion, responses on the active lever have no consequences during a 20 s time-out period. Inactive lever presses also have no consequences, and are were recorded.

After the last day of self-administration, rats experience daily 3-h extinction sessions. On the first session, a catheter is connected to the swivel; however, no drug is administered. On subsequent sessions, rats are placed into the chamber without being attached to the swivel. Throughout extinction training, the house light signals the initiation of the session and remains illuminated during the session. Responses on either the active or inactive lever are recorded, but resulted in no programmed consequences (i.e., no infusion and no CS presentation). Animals continue under extinction conditions until they reached a criterion of a minimum of 10 days and <25 lever presses per session for two consecutive days.

Following extinction, rats undergo four reinstatement tests, using a counterbalanced, within subjects design, with a minimum of 2 days of extinction between each test. Immediately prior to each reinstatement test, the rats receive either intracranial vehicle or B/M. During the first two reinstatement tests, rats are placed into the chambers for 3 h, during which the house light is illuminated and each active lever press results in a 5 s CS presentation in the absence of any drug reinforcement, followed by a 20 s time out period. For heroin-primed reinstatement tests, a single, non-contingent dose of heroin (0.25 mg/kg, s.c.) is administered immediately prior to the rat entering the chamber for a 3 h session, during which the house light is illuminated and lever responses have no programmed consequences (i.e., no CS presentation).

Lever responses and heroin intake during self-administration, as well as lever responses during extinction, CS-induced, and heroin-primed reinstatement testing are assessed using analysis of variance (ANOVA). Post-hoc analyses are conducted using Tukey tests and analyses are considered statistically significant at P<0.05. All data are presented as mean±SEM. Correlations are generated using Pearson's product correlations to test for correlations of responses between the different reinstatement tests.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. (canceled)

2. (canceled)

3. A method of reducing an elevated amount of ΔFos in a subject having an addiction, comprising administering to the subject a therapeutically effective amount of a pharmaceutical composition that comprises digestive enzymes, wherein ΔFos is elevated in the subject due to the addiction compared to a subject without the addiction.

4. A method of decreasing an amount or a length of dendritic spines in nerve cells of a subject having an addiction, comprising administering to the subject a therapeutically effective amount of a pharmaceutical composition that comprises digestive enzymes, thereby decreasing the amount or the length of the dendritic spines in nerve cells of the subject to an amount that is lower than prior to administration of the pharmaceutical composition.

5. The method of claim 4, wherein the digestive enzymes comprise an amylase, a lipase, and a protease.

6-9. (canceled)

10. The method of claim 4, wherein a dose of the pharmaceutical composition comprises about 4, about 5, about 6, about 7, about 8, or about 9 capsules, tablets, or sachets.

11-13. (canceled)

14. The method of claim 4, wherein the addiction comprises a drug addiction, wherein the drug comprises an opiate.

15. (canceled)

16. The method of claim 14, wherein the opiate comprises a natural opiate, a synthetic opiate, or a combination thereof.

17-20. (canceled)

21. The method of claim 4, wherein the addiction comprises an alcohol addiction.

22-29. (canceled)

30. The method of claim 4, wherein the pharmaceutical composition comprises coated digestive enzyme particles.

31. (canceled)

32. The method of claim 30, wherein the coated digestive enzyme particles comprise (i) a core that comprises the digestive enzymes and (ii) a coating.

33. The method of claim 32, wherein the coating comprises an enteric coating.

34. The method of claim 32, wherein the coating comprises a lipid.

35. (canceled)

36. (canceled)

37. The method of claim 34, wherein the lipid comprises a pharmaceutical grade lipid.

38. The method of claim 37, wherein the pharmaceutical grade lipid comprises a fully-hydrogenated soybean oil.

39-45. (canceled)

46. The method of claim 34, wherein the lipid comprises monoglycerides, diglycerides, and triglycerides.

47. The method of claim 34, wherein the lipid comprises a hydrogenated lipid, a partially saturated lipid, or a combination thereof.

48. The method of claim 34, wherein the lipid comprises a soy lipid.

49. The method of claim 48, wherein the soy lipid comprises a hydrogenated soy lipid.

50-53. (canceled)

54. The method of claim 4, wherein the pharmaceutical composition is administered to the subject orally.

55-59. (canceled)

60. The method of claim 4, wherein
the subject is administered one or more doses of the pharmaceutical composition.

61. The method of claim 60, wherein a dose of the pharmaceutical composition comprises from about 100 to about 9,000 mg of the digestive enzymes.

62-94. (canceled)

95. A pharmaceutical composition for treatment of an addiction in a subject in need thereof, wherein the pharmaceutical composition comprises encapsulated, coated digestive enzyme particles, wherein the coated digestive enzyme particles comprise (i) a core that comprises digestive enzymes that comprise a protease, an amylase, and a lipase, and (ii) a coating.

96-119. (canceled)

120. The method of claim 5, wherein a dose of the pharmaceutical composition comprises from about 650,000 U.S.P. units to about 1.5×10⁶ U.S.P. units of the protease.

121. The method of claim 120, wherein a dose of the pharmaceutical composition comprises from about 650,000 U.S.P. units/unit dose to about 1.5×10⁶ U.S.P. units/unit dose of the protease.

122. The method of claim 4, wherein the subject to be treated has a blood level of phenylalanine that is two-fold less than 2 mg/dL or 120 micromolar.

123. The method of claim 4, wherein a dose of the pharmaceutical composition is formulated as a sachet.
